(19) United States
(12) Patent Application Publication
(10) Pub. No.: US 2015/0127005 A1
(43) Pub. Date: May 7, 2015

(54) CLAVICLE REAMER

(71) Applicant: DEPUY SYNTHES PRODUCTS, LLC, Raynham, MA (US)

(72) Inventor: Joshua MCMANUS, Downingtown, PA (US)

(21) Appl. No.: 14/532,454

(22) Filed: Nov. 4, 2014

Related U.S. Application Data
(60) Provisional application No. 61/900,398, filed on Nov. 6, 2013.

(51) Int. Cl. A61B 17/16

(52) U.S. Cl. CPC A61B 17/1691
USPC 606/80

(57) ABSTRACT

A device for reaming a bone includes an elongated shaft extending along a first longitudinal axis from and first end to a second end, the shaft being sufficiently longitudinally flexible to enable it to be passed through a path having a curvature of a target bone and a reaming head extending along a second longitudinal axis from the second end to a third free end, wherein the first longitudinal axis is parallel to and laterally offset from the second longitudinal axis so that, upon rotation of the shaft, the reaming head rotates eccentrically with respect to the first longitudinal axis.

### CROSS REFERENCE TO RELATED APPLICATIONS

This application relates to U.S. patent application Ser. No. 61/900,398, filed Nov. 6, 2013; all applications are herein incorporated by reference in their entireties.

### BACKGROUND INFORMATION

The majority of clavicle fractures are treated with either non-operative conservative treatment or internal fixation via a superior or anterior bone plate. Fractures treated via bone plates generally go on to heal but patients often complain of soft tissue irritation from tissue stripping around the bone during the surgical procedure. In addition, many patients dislike the prominence of the bone plate on the bone and it is not uncommon for patients to have the plate removed with a second surgical procedure after healing. One alternative to the use of bone plates for clavicle fixation is an intramedullary nail which reduces the soft tissue stripping required and eliminates the prominence experienced with bone plates. However, current intramedullary nails are generally incapable of accommodating the substantially “S”-shaped curvature of the clavicle. Thus, only a small subset of clavicle fractures can be treated with today's clavicle nail technologies. A particularly difficult challenge relating to clavicle nailing involves drilling/reaming a uniform path along the “S”-shaped medullary canal of the clavicle. Previous drilling/reaming concepts at times resulted in the piercing of the cortical wall of the clavicle, thereby further weakening the bone and putting the surrounding neural and vascular structures at risk.

### SUMMARY OF THE INVENTION

The present invention is directed to a device for reaming a bone, comprising an elongated shaft extending along a first longitudinal axis from and first end to a second end, the shaft being sufficiently longitudinally flexible to enable it to be passed through a path having a curvature of a target bone and a reaming head extending along a second longitudinal axis from the second end to a third free end, wherein the first longitudinal axis is parallel to and laterally offset from the second longitudinal axis so that, upon rotation of the shaft, the reaming head rotates eccentrically with respect to the first longitudinal axis.

The present invention is further directed to a method for reaming a bone, comprising inserting a reaming device into a medullary canal of a bone, the reaming device including an elongated, longitudinally flexible shaft extending along a first longitudinal axis and a reaming head connected to the shaft, the reaming head extending along a second longitudinal axis laterally offset from and parallel to the first longitudinal axis and rotating the shaft about the first longitudinal axis to rotate the reaming head within the medullary canal, the reaming head rotating eccentrically with respect to the first longitudinal axis so that, as the reaming head enters a curve in the medullary canal, the reaming head skives off a cortical wall of the bone and remains within the medullary canal following a curvature of the bone.

### BRIEF DESCRIPTION OF THE DRAWINGS

FIG. 1 shows a perspective view of an exemplary device according to the invention;

FIG. 2 shows a side view of the device of FIG. 1;

FIG. 3 shows the device of FIG. 1 in a first operative configuration; and

FIG. 4 shows the device of FIG. 1 in a second operative configuration.

### DETAILED DESCRIPTION

The present invention may be further understood with reference to the following description and the appended drawings, wherein like elements are referred to with the same reference numerals. The exemplary embodiments of the present invention relate to a system and method for the repair of a fractured, shattered or otherwise damaged clavicle using an intramedullary nail. An exemplary clavicle reamer according to the present invention may be used for drilling within the clavicle an elongated channel sized, shaped and oriented to receive an intramedullary nail. An exemplary reamer according to the invention includes an elongated substantially longitudinally flexible, torsionally rigid shaft extending from a proximal end connected to a rotary mechanism, as those skilled in the art will understand, to a distal end having a reamer. A central longitudinal axis of the shaft is laterally offset from a central longitudinal axis of the reamer head by a predetermined distance such that rotation of the shaft rotates the reamer head eccentrically about an axis offset from the central longitudinal axis of the shaft, as will be described in greater detail below. The offset movement of the reamer head, along with the longitudinal flexibility of the elongated shaft permits the clavicle reamer to skive off of a cortical wall of the clavicle as the clavicle is being reamed so that the reamer head is guided along the curvature of the medullary canal without damaging the cortical bone. That is, the skiving movement prevents the reamer from entering the cortical portion of the bone and instead guides the reamer along a spongy portion of the bone radially within the cortical bone to prevent the reamer from penetrating laterally through the bone. It is noted that although the exemplary embodiment is described with respect to a clavicular fixation procedure, the exemplary reamer according to the invention may be employed with any other short bone or any long bone including a curved medullary canal to ream a channel within the medullary canal following the curve of a wall of the medullary canal, thereby reducing or eliminating the likelihood of the reamer extending out of a lateral wall of the bone during the reaming procedure.

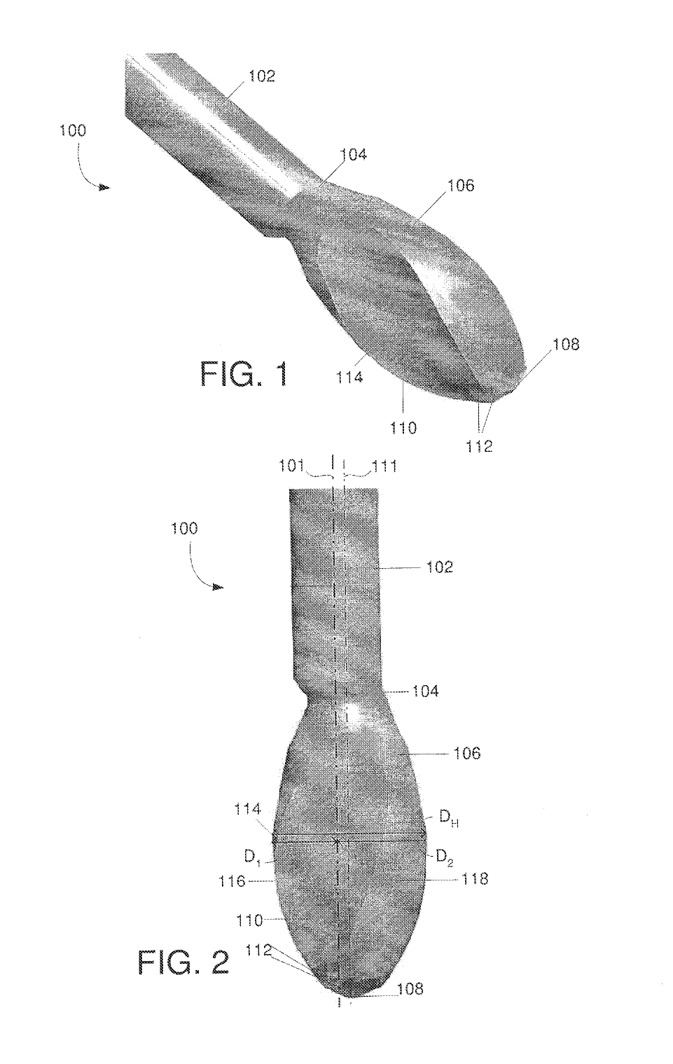

As shown in FIGS. 1-2, a reaming device 100 according to an exemplary embodiment of the present invention comprises a shaft 102 extending along a first longitudinal axis 101 from a first end (not shown) connectable to any known rotary device to rotate the shaft 102 so that rotation is transmitted along the shaft 102 to a second end 104. A reamer head 106 is connected to the second end 104 of the shaft 102 and extends from the second end 104 to a free end 108 of the reamer head 106. In an exemplary embodiment, the shaft 102 and reamer head 106 are integrally formed. In another embodiment, the shaft 102 and reamer head 106 may be formed separately and permanently bonded to one another via welding or another bonding means known in the art. The shaft 102 is formed of a biocompatible material and dimensioned to have a longitudinal flexibility selected to match the anatomy through which the shaft is to be passed as would be understood by those skilled in the art. For example, a shaft 102 for use in a clavicle may be formed of stainless steel. The material of the shaft 102 may be selected to impart a predetermined longitudinal flexibility to the shaft 102. In one example, the shaft 102 must be capable of elastically bending along a curve with a radius between 0.03 m and 1 m. A diameter of the shaft 102 may range between 0.8 mm and 7 mm. to conform to the spatial restrictions of a particular procedure and/or anatomical location. A ratio of the diameter of a medullary canal of a bone to be reamed and a diameter of the shaft 102 may be between 6:1 and 2:1. Furthermore, the shaft 102 is formed to have a torsional stiffness sufficient to transmit rotation applied at the first end to the second end 104 as would be understood by those skilled in the art. In an exemplary embodiment, the longitudinal flexibility of the shaft 102 is selected to conform to the degree of deviation required as the reamer head 106 moves along the medullary canal without penetrating a cortical wall of the bone. For example, the degree of longitudinal flexibility is selected to be made greater in systems designed for use in short bones (e.g., the clavicle) and marginally smaller in systems designed for use in longer bones (e.g., the proximal humerus). Furthermore, the degree of flexibility may correspond to the curvature of a particular bone to be treated. The reamer head 106 may be formed of the same material as the shaft 102 or, in another embodiment, may be formed of a material having a greater rigidity than the shaft 102. The reamer head 106 may have a length of 2-30 mm and a diameter of 2-18 mm, the dimensions being selected to conform to the anatomy of a target bone. In an exemplary embodiment, the reamer head 106 may be formed of stainless steel, titanium, titanium alloys, stainless steel alloys, cobalt chrome or nitinol. In an exemplary embodiment, the reamer head 106 includes two cutting flutes 110 extending therealong to the free end 108 along a path that is, for example, diagonal or helical. The number of the cutting flutes 110 may be modified to conform to requirements of any procedure, for example, with larger reamer heads (e.g., for larger bones) including a greater number of flutes and smaller reamer heads (e.g., for smaller bones) including a lesser number of flutes as would be understood by those skilled in the art. In another embodiment (not shown), the device 100 may include anywhere from one to four flutes 110 distributed evenly over an outer surface of the reamer head 106. The free end 108 of the reamer head 106 may include one or more sharpened cuts 112 to aid in the penetration of the bone.

An axial length of the reamer head 106 may be selected to conform to the requirements of a particular procedure, with shorter lengths of the reamer head 106 providing added flexibility to the device 100. The reamer head 106 has an elongated oval or “football” shape. It is noted, however, that the reamer head 106 may be formed with another shape without deviating from the scope of the invention including, but not limited to, spherical, cylindrical and hourglass. In the exemplary embodiment shown in FIGS. 1-3, a diameter of the reamer head 106 tapers from a first smaller diameter at the second end 104 to a maximum diameter D_{H} at a central portion 114 and back down to a smaller diameter at the free end 108. This exemplary configuration provides a slightly rounded front end to reduce trauma and aid in guiding the reamer along a curved path while reducing heat generated by the cutting head during operation. In an exemplary embodiment, a ratio of the diameter D_{H} to a diameter D_{S} of the shaft 102 is 2:1, although other ratios may be used without deviating from the scope of the invention (e.g., between 5:4 and 5:1).

A central axis 111 of the reamer head 106 extends along a path parallel to and laterally offset from the first longitudinal axis 101 by approximately 0.05-2 mm. Thus, a first portion 116 of the reamer head 106 on a first side of the first longitudinal axis 111 extends laterally away from the axis 101 by a first distance D₁ and a second portion 118 of the reamer head 106 located on a second side of the first longitudinal axis 101 extends laterally away from the axis 101 by a second distance D₂, wherein D₁<D₂. Thus, when the shaft 102 is rotated about the first longitudinal axis 101 within a bone, reaming is generally concentrated in portions of bone contacted by the second portion 118. That is, as the reaming device 100 is rotated within a bone, a channel formed in the bone is centered about the first longitudinal axis 101 but has a diameter approximately equal to the distance D₂. This eccentric mounting of the reamer head 106 causes the reamer head 106 to “wobble” about the first axis 101 so that, when encountering a curve in the medullary canal, the reamer head 106 skives off a cortical wall of the clavicle and, as the reaming device 100 is further inserted into the canal, the reamer head 106 is redirected back toward an axis of the medullary canal without damaging the cortical bone, as will be described in greater detail in the description of the exemplary method below.

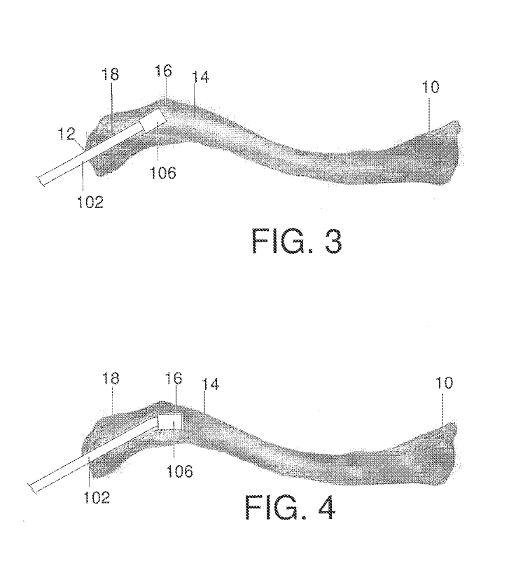

As shown in FIGS. 3-4, in use the reaming device 100 is inserted into a lateral end 12 of a clavicle 10 and advanced into a medullary canal thereof along a central axis of the canal. Due to the S-shape of the clavicle, as the reaming device 100 is inserted axially into the clavicle, the reamer head 106 penetrates a spongy tissue portion 14 heading toward an outer cortical wall 16 of the bone, as shown in FIG. 3. However, as resistance to further movement of the reamer head 106 along this path increases due to contact with compact tissue of the cortical wall 16, the eccentric reamer head 106 skives off the cortical wall 16 rather than penetrating the cortical wall and moves along the cortical wall to follow the path of the medullary canal. Specifically, the wobbling movement of the reamer head 106, along with the longitudinal flexibility of the shaft 102 permits the reamer head 106 to slide tangentially along the curve of the cortical wall 16 remaining in the spongy bone 14 as the reamer is further advanced into the bone, as shown in FIG. 4. Thus, a channel 18 reamed in the clavicle 10 has a shape substantially matching an S-shape of the clavicle 10.

As described above, the exemplary system according to the present invention permits the reaming of a channel 18 conforming to the curvature of the clavicle 10. The curved channel 18 may then be used to guide the insertion of a correspondingly curved intramedullary nail (not shown) into the clavicle 10 to permit internal fixation thereof. In an exemplary procedure, the reamer is advanced in one of a medial to lateral direction and a lateral to medial direction. The reaming device 100 may be used to ream a complete length of the clavicle 10 or any portion of the clavicle 10. In another embodiment, the reaming device 100 may remain implanted within the clavicle to provide intramedullary fixation.

The exemplary system and method disclosed above may be particularly useful for the drilling/reaming of any short or long bone having a curvature. For example, the reaming device 100 may be used to ream a proximal humerus with a lateral insertion, the tibia, or any other bone in the body. The exemplary system may be employed in a minimally invasive bone fixation procedure where, for example, only one incision is to be made adjacent a bone fracture site. In accordance with an exemplary method according to the present invention, a minimally invasive incision is formed through the skin adjacent an end of a short or long bone.

It will be apparent to those skilled in the art that various other modifications and variations can be made in the structure and the methodology of the present invention, without departing from the spirit or scope of the invention. Thus, it is intended that the present invention cover the modifications and variations of this invention provided that they come within the spirit of the appended claims and their equivalents.

What is claimed is:

1. A device for reaming a bone, comprising:
an elongated shaft extending along a first longitudinal axis from and first end to a second end, the shaft being sufficiently longitudinally flexible to enable it to be passed through a path having a curvature of a target bone; and
a reaming head extending along a second longitudinal axis from the second end to a third free end, wherein the first longitudinal axis is parallel to and laterally offset from the second longitudinal axis so that, upon rotation of the shaft, the reaming head rotates eccentrically with respect to the first longitudinal axis.

2. The device of claim 1, wherein the reaming head is integrally formed with the shaft.

3. The device of claim 1, wherein the reaming head is formed separately from the shaft.

4. The device of claim 3, wherein the reaming head is permanently bonded to the shaft.

5. The device of claim 1, wherein the reaming head includes a cutting flute extending along an outer surface thereof.

6. The device of claim 5, wherein the cutting flute extends in one of a diagonal and helical pattern over the outer surface of the reaming head.

7. The device of claim 1, wherein the first longitudinal axis is laterally offset from the second longitudinal axis by a distance of 0.05-2 mm.

8. The device of claim 1, wherein a longitudinal cross-section of the reaming head is oval.

9. The device of claim 8, wherein a ratio of a maximum diameter of the reaming head to a diameter of the shaft is 5:1.

10. The device of claim 1, wherein the shaft is longitudinally elastically deformable within a predetermined range.

11. The device of claim 10, wherein the shaft is deflectable in a plane extending orthogonal to the first longitudinal axis along a curve with a radius of 0.8 m -7 m.

12. The device of claim 1, wherein the device is sized and shaped to ream an medullary canal of a clavicle.

13. The device of claim 1, wherein the elongated shaft is connected to a rotary mechanism to rotationally driving the reaming device.

14. A method for reaming a bone, comprising:
inserting a reaming device into a medullary canal of a bone, the reaming device including an elongated, longitudinally flexible shaft extending along a first longitudinal axis and a reaming head connected to the shaft, the reaming head extending along a second longitudinal axis laterally offset from and parallel to the first longitudinal axis; and
rotating the shaft about the first longitudinal axis to rotate the reaming head within the medullary canal, the reaming head rotating eccentrically with respect to the first longitudinal axis so that, as the reaming head enters a curve in the medullary canal, the reaming head skives off a cortical wall of the bone and remains within the medullary canal following a curvature of the bone.

15. The method of claim 14, further comprising the step of actuating a rotary mechanism coupled to the elongated shaft to rotate the shaft.

16. The method of claim 14, wherein the flexible shaft is longitudinally elastically deformable within a predetermined range.

17. The method of claim 14, wherein the reaming head is one of integrally formed and bonded to the shaft.

18. The method of claim 14, wherein the bone is a clavicle.
